(19) United States
(12) Plant Patent Application Publication
(10) Pub. No.: US 2002/0199230 P1
(43) Pub. Date: Dec. 26, 2002

(54) Guzmania plant named 'Deplagnum'

(76) Inventor: Ronald Bunnik, Pijnacker (NL)

Correspondence Address: FOLEY & LARDNER, Suite 500, 3000 K Street, N.W., Washington, DC, 20007-5109, US

(21) Appl. No.: 10/004,347

(22) Filed: Dec. 6, 2001

(30) Foreign Application Priority Data
Apr. 25, 2001 (EP) 2001/0626

(51) Int. Cl. A01H 5/00

(52) U.S. Cl. PLT/371

(57) ABSTRACT

A new and distinct Guzmania plant named ‘Deplagnum’ characterized by big inflorescence size; purple inflorescence; flat top of inflorescence; and dark, wide green leaves.

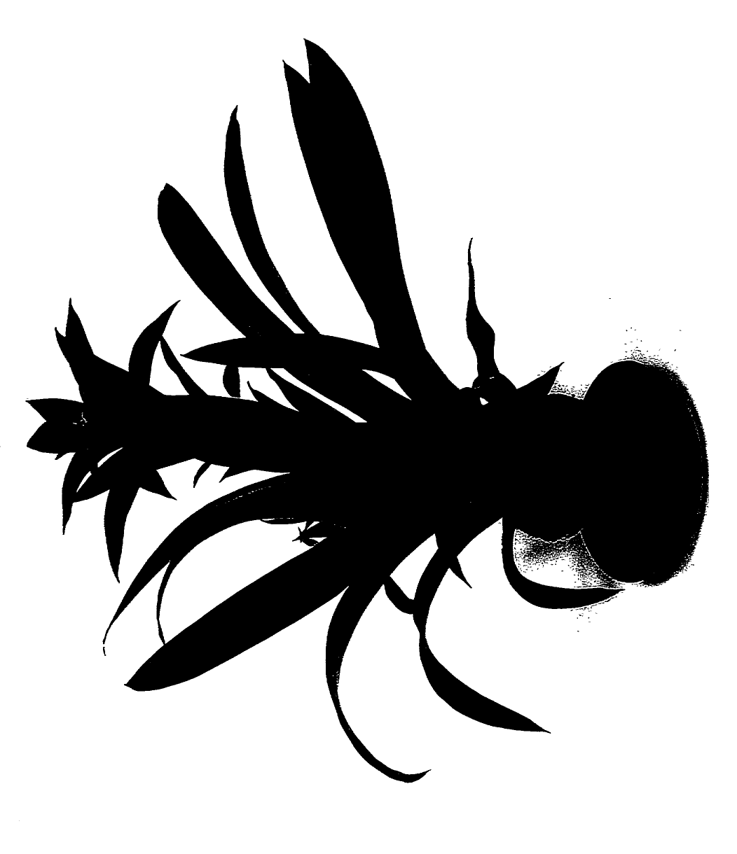

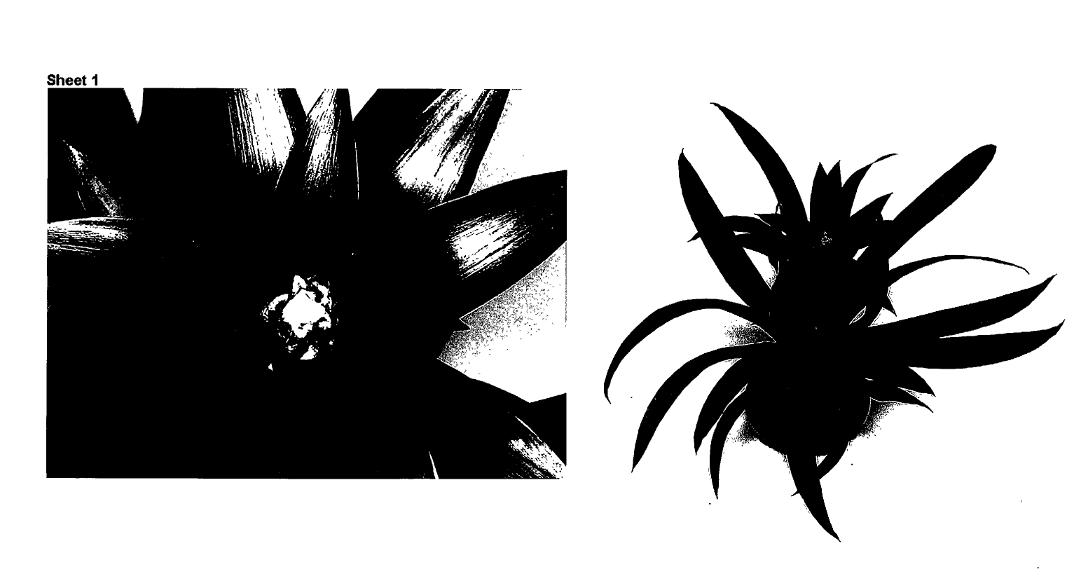

### BACKGROUND OF THE INVENTION

The present invention comprises a new and distinct cultivar of Guzmania plant, hereinafter referred to by the cultivar name ‘Deplagnum’. The genus Guzmania is a member of the family Bromeliaceae.

Guzmania comprise a genus of over 100 species of herbaceous evergreen perennials suitable for cultivation in the home or under glass. Guzmania are predominantly epiphytic with a few terrestrial species and are native to the tropics. For the most part the species vary in diameter from 7 or 8 inches to 3 or 4 feet and have rosettes of glossy, smooth edged leaves.

Floral bracts of Guzmania frequently have brilliant colors and may last for many months. The range of flower colors for Guzmania is generally from yellow through orange but may also include flame red and red-purple. White or yellow, tubular, three petalled flowers may also appear on a stem or within the leaf rosette but are usually short lived.

Guzmania may be advantageously grown as potted plants for greenhouse or home use. Desirably the plants are shaded from direct sunlight during the spring to autumn period, the central vase-like part of the leaf rosette is normally filled with water.

Guzmania is native to tropical America. Leaves of the Guzmania are usually formed as basal rosettes which are stiff and entire and in several vertical ranks. Guzmania have terminal spikes or panicles which are often bracted with petals united in a tube about as long as the calyx.

Asexual propagation of Guzmania is frequently done through the use of tissue culture practices. Propagation can also be from off-shoots which are detached from the mother plant, and may be grown in an appropriate soil or bark mixture.

The new cultivar ‘Deplagnum’ is the product of a planned breeding program and was originated by the inventor Ronald Bunnik, as a mutant from an unknown parent during 1995 in Pijnacker, The Netherlands. The selection comprising the new variety was chosen after commencement of flowering in 1995.

The new cultivar was asexually propagated by taking cuttings by the inventor in Pijnacker, The Netherlands in 1995. Asexual propagation by tissue culture was initiated in 1999 by Deroose Plants, in Evergem, Belgium. Continuous asexual propagation has demonstrated that the combination of characteristics as herein disclosed for the new cultivar ‘Deplagnum’ are firmly fixed and are retained through successive generations of asexual reproduction. The new cultivar reproduces true to type.

### BRIEF DESCRIPTION OF THE INVENTION

The following traits have been repeatedly observed and are determined to be basic characteristics of ‘Deplagnum’ which in combination distinguish this Guzmania as a new and distinct cultivar:

1. big inflorescence size;

2. purple inflorescence;

3. flat top inflorescence;

4. dark and wide green leaves

‘Deplagnum’ has not been observed under all possible environmental conditions. The phenotype of the new cultivar may vary significantly with variations in environment such as temperature, light intensity, and day length without any change in genotype.

Of the many commercial cultivars known to the present inventor, the most similar in comparison to ‘Deplagnum’ is the parental cultivar ‘Indian’. In comparison to ‘Indian’, the inflorescence of ‘Deplagnum’ is flatter on the top, is much bigger and darker colored. Guzmania ‘Deplagnum’ also has darker green and wider leaves than ‘Indian’. The flower shape of ‘Deplagnum’ is more compact than the flower shape of ‘Indian’.

### BRIEF DESCRIPTION OF THE DRAWING

The accompanying photographic illustration shows a 19-month-old ‘Deplagnum’ plant following growth under appropriate growing conditions, with colors being as true as possible with illustrations of this type. The first drawing shows a side view of the plant (right) and a close up of the inflorescence (left). The second drawing shows a side view of the plant.

### DETAILED BOTANICAL DESCRIPTION

The following observations, measurements and values describe plants grown in Pijnacker, The Netherland under greenhouse conditions which closely approximate those generally used in horticultural practice. ‘Deplagnum’ is grown in a commercial greenhouse under 21 degrees Celsius day and night. No artificial lighting or photoperiodic treatments are conducted but ‘Deplagnum’ is forced into flowering by adding acethylene. Highest temperature resistance is 40 degrees Celsius, the lowest 5 degrees Celsius. Direct sunlight has to be avoided because it causes burning of the leaves. The following fertilizer is added: 1 part nitrogen, 0.5 parts phosphor. 3 parts Kalium and 0.2 parts Magnesium. Water should not contain too much salts. From the start of tissue culture it takes five years to produce a commercial plant. The amount of time needed to produce a flower depends on the amount of acethylene is added. The flowers have a tenability of up to six months depending on the environment they are grown and kept in.

Color references are made to the Royal Horticultural Society Colour Chart (RHS), except where general colors of ordinary significance are used.

### CLASSIFICATION:

Commercial.—Guzmania c.v. Deplagnum

PARENTAGE: Unknown parentage

PROPAGATION: Tissue culture

### PLANT:

Form.—Upright, leaves in basal rosette

Height.—Average 42 cm

Diameter.—Average 50 cm

Growth Habit.—Upright, growth moderate

### FOLIAGE:

Size.—Leaves have an average length of 31 cm and an average width of 4 cm (measured at the middle of a leaf).

Shape.—Linear, tips acute

Surface Texture.—Smooth, glossy

Color.—Leaf color upper side dark yellow-green, closest too but slightly darker than RHS 147 A. Leaf color under side yellow-green RHS 147 A.

### BRACTS:

Length/Width/Color.—Length average 9 cm; width average 3.2 cm (measured at ⅓from the base), tips acute, upper side color greyed-purple closet to RHS 187 B (slightly more reddish), under side color yellow-green RHS 187 C. Lower bracts tipped green RHS 147 A to almost entirely green (only greyed-purple at the base).

General shape/Arrangement.—Broad lanceolate, arranged alternately, tips horizontal to slightly bent downwards in an average angle of 0° (=horizontal) to 40°.

Number.—Terminal Bracts: Average 12 Primary Bracts: Average 7 Scape Bracts: Average 7

Texture.—Smooth, glossy

Margin.—Entire

Scape.—Length average 25 cm; width average 1 cm (measured approximately ⅓from the base of the primary bracts)

BRANCH SPIKES: Spikes (scapes) unbranched

INFLORESCENCE: No observations made to date

REPRODUCTIVE ORGANS: No flowers observed to date

ROOTS: Thin, very well-branched, strong to moderately strong, color brown RHS 199 C

SEED CHARACTERISTICS: No fruits or seeds observed to date

I claim:

1. A new and distinct Guzmania plant named ‘Deplagnum’ as described and illustrated herein.
